(19) United States
(12) Patent Application Publication
(10) Pub. No.: US 2019/0233350 A1
(43) Pub. Date: Aug. 1, 2019

(54) PROCESS FOR CONVERSION OF HYDROCARBON FEED TO C2 UNSATURATED HYDROCARBONS AND SYNGAS COMPOSITION USED FOR MULTIPLE APPLICATIONS

(71) Applicant: SABIC GLOBAL TECHNOLOGIES B.V., Bergen op Zoom (NL)

(72) Inventors: Krishnan SANKARANARAYANAN, Missouri City, TX (US); Pankaj Singh GAUTAM, Evansville, IN (US); Aghaddin Khanlar MAMEDOV, Sugar Land, TX (US); Sreekanth PANNALA, Sugar Land, TX (US); Balamurali Krishna RAMACHANDRAN NAIR, Sugar Land, TX (US); Istvan LENGYEL, Lake Jackson, TX (US); David WEST, Bellaire, TX (US)

(21) Appl. No.: 16/331,683

(22) PCT Filed: Sep. 12, 2017
(86) PCT No.: PCT/US2017/051115

§ 371 Date: Mar. 8, 2019

Related U.S. Application Data
(60) Provisional application No. 62/395,544, filed on Sep. 16, 2016.

(51) Int. Cl. C07C 4/02; C07C 5/09; C07C 2/82; C07C 11/04; C07C 11/24; B01D 53/047; C01B 3/24; C01B 3/36; C01B 3/38

(52) U.S. Cl. CPC C07C 4/025; C07C 5/09; C07C 2/82; C07C 11/04; C07C 11/24; B01D 53/047; C01B 2203/0272; C01B 3/36; C01B 3/38; C07C 2523/44; C07C 2523/60; C01B 2203/0238; C01B 2203/0255; C01B 3/24

(57) ABSTRACT

Integrated processes for the conversion of hydrocarbons to C2 and C3 unsaturated hydrocarbons include combustion and cracking of hydrocarbons, dry oxidative reforming of methane, and catalytic hydrogenation of acetylene. Reactive products formed among the integrated processes may be distributed and recycled among the processes for the conversion of the hydrocarbon feedstock.

### FIELD OF DISCLOSURE

The present disclosure relates to the production of hydrocarbons. More specifically, the disclosure relates to the conversion of hydrocarbons to C2 (two carbons) unsaturated hydrocarbons and syngas products.

### BACKGROUND

Complex hydrocarbons, including olefins such as acetylene and ethylene, are useful in a wide range of products and applications. Traditional methods of converting lower molecular weight carbon-containing molecules to higher molecular weights are numerous. Unsaturated hydrocarbons have been prepared via thermal pyrolysis in the conversion of natural gas condensates and petroleum distillates, which include methane, ethane, and larger hydrocarbons. One process, commonly referred to as cracking, utilizes considerable amounts of energy to provide the unsaturated product. The most prevalent methods involve oxidative coupling, partial oxidation, or pyrolysis. The generation of side products, such as carbon dioxide and syngas, in oxidative pyrolysis processes can affect the overall unsaturated hydrocarbon product.

### SUMMARY OF THE DISCLOSURE

As described in more detail herein, the present disclosure provides processes, apparatuses, and systems for the production of C2 (two carbons) unsaturated hydrocarbons and syngas. Integrated processes of thermal pyrolysis, methane dry oxidative reforming, and catalytic hydrogenation of acetylene are described. Aspects of the present disclosure allow for a generated carbon dioxide product to undergo an oxidative dry reforming process. Consumption of the generated carbon dioxide may shift the molar ratio of the reaction products of the integrated processes to increase the amount of unsaturated hydrocarbon and syngas produced.

In an aspect, a method may comprise: subjecting a hydrocarbon feedstock to an oxidative pyrolysis process to form at least acetylene, a carbon dioxide product, and a first syngas product; hydrogenating the acetylene to form one or more of ethylene and a hydrogenation product; and converting at least a portion of the formed carbon dioxide product to at least a second syngas product through an oxidative dry reforming process, wherein the first syngas product approaches a ratio of 2:1 hydrogen gas to carbon monoxide and wherein the second syngas product approaches a ratio of 1.5 hydrogen gas to carbon monoxide.

### BRIEF DESCRIPTION OF THE FIGURES

The accompanying figures, which are incorporated in and constitute a part of this specification, illustrate several aspects and together with the description serve to explain the principles of the disclosure.

FIG. 1 shows a reaction scheme for the integration of a methane-to-acetylene conversion reaction with a methane dry oxidative reforming reaction.

FIG. 2 shows a reactor block diagram for the integration of a methane-to-acetylene conversion reaction with acetylene hydrogenation and a methane dry oxidative reforming reaction.

FIG. 3 shows a process block diagram for the integration of a methane-to-acetylene conversion reaction with acetylene hydrogenation and a methane dry oxidative reforming reaction.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims.

### DETAILED DESCRIPTION

Unsaturated C2-C3 hydrocarbons may be prepared by the oxidative thermal conversion of various hydrocarbons Often, the unsaturated hydrocarbons are prepared by cracking a mixture of saturated C2-C3 hydrocarbons. The processes associated with cracking generally consume large amounts of energy (e.g., about 45 kilocalories of energy per mole (kcal/mol) methane) to achieve conversion of the hydrocarbons. Further, the process of cracking may be accompanied by the generation of significant amounts of carbon dioxide in addition to the desired C2-C3 (two to three carbons) unsaturated carbon product. Additional separation processes are needed to isolate the desired unsaturated C2-C3 product form the carbon dioxide. The present disclosure may include one or more of combustion/cracking of methane, dry oxidative reforming of methane, and catalytic hydrogenation of generated acetylene to utilize the carbon dioxide side product and improve carbon efficiency of the overall process.

In various aspects, the present disclosure provides an integration of processes for the conversion of saturated hydrocarbons to unsaturated hydrocarbons. The integrated processes may combine oxidative thermal conversion of a hydrocarbon feedstock to acetylene with an acetylene hydrogenation and a process of dry oxidative methane reforming (also described as methane oxidative dry reforming) to utilize carbon dioxide generated during the oxidative thermal conversion. Specifically, a method of producing unsaturated C2 hydrocarbons may comprise subjecting a hydrocarbon feedstock to an oxidative pyrolysis process to form at least acetylene, a carbon dioxide product, and a first syngas product.

Generated acetylene may be then be hydrogenated to form one or more of ethylene and a hydrogenation byproduct. At least a portion of the formed carbon dioxide product may be converted through an oxidative dry reforming process to form at least a second syngas product. The first syngas product approaches a ratio of 2:1 hydrogen gas to carbon monoxide and the second syngas product approaches a ratio of 3:2 hydrogen gas to carbon monoxide. By utilizing the carbon dioxide product in a separate process to generate syngas, the syngas ratio may be improved compared to syngas generated in a system recycling the carbon dioxide to the cracking reactor or separating carbon dioxide from the final product. Carbon dioxide may thus be converted to useful chemicals, thereby optimizing carbon dioxide utilization and improving carbon efficiency.

A hydrocarbon feedstock may be subjected to an oxidative pyrolysis process to form unsaturated C2-C3 hydrocarbons and to form carbon dioxide and syngas side products. The products from the oxidative pyrolysis process may be separated according to any number of appropriate separation processes. Acetylene isolated from the oxidative pyrolysis products may be hydrogenated to form ethylene according to hydrogenation processes well known in the art. The separated carbon dioxide may be diverted to a separate process of methane dry oxidative reforming to provide an additional syngas product.

During high temperature methane partial oxidation, cracking of methane and following acetylene hydrogenation the following reactions 1-4 may occur:

CH₄+2O₂=CO₂+2H₂O (1)

CH₄+1.5O₂=CO+2H₂O (2)

2CH₄=C₂H₂+3H₂ (3)

C₂H₂+H₂=C₂H₄ (4)

Reactions 1-3 may proceed in a cracking reactor and comprise the cracking process or phases. Reaction 4 may occur separately as a catalytic hydrogenation of acetylene to ethylene.

Conventionally, an overall equation summarizing equations (1-4) for the conversion of methane to ethylene may be expressed as (5):

4CH₄+3O₂=C₂H₄+3H₂+CO+CO₂+3H₂O ΔH=−52 kcal/mol (5)

Reaction 5 may describe the overall product mixture stream after the combustion/cracking reactor stage. The ethylene (C₂H₄) and carbon dioxide (CO₂) molar ratio (as weight) in the product mixture stream may be between about 1.3 and 1.45. While carbon monoxide (CO) and hydrogen gas (H₂) may be useful as reagents for syngas conversion reactions (such as syngas to methanol or syngas to olefins), side products, such as carbon dioxide, may result in a decrease of selectivity of the overall process and a decrease in carbon efficiency of the process.

Methods of the present disclosure describe a separation of carbon dioxide from the product mixture stream and conversion of the carbon dioxide via dry oxidative reforming of methane. After separation of the carbon dioxide, acetylene may be hydrogenated by a syngas mixture (H₂+CO) to ethylene. After the hydrogenation of acetylene to ethylene and separation of the desired ethylene, the syngas mixture may be collected for further chemicals, such as methanol production.

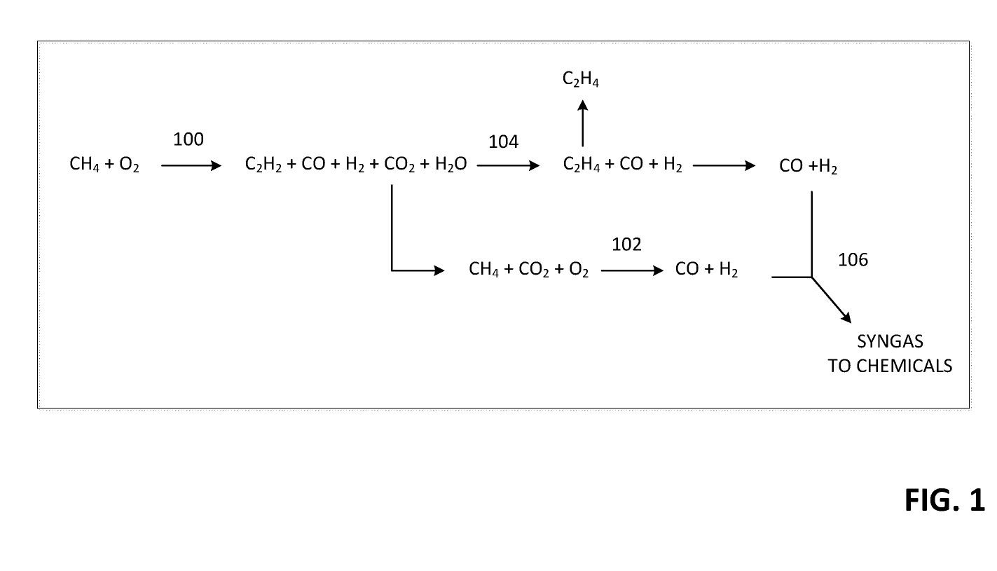

As an illustrative example, FIG. 1 presents multiple reaction processes combined to improve carbon efficiency. Methane may be reacted with oxygen to provide acetylene, carbon monoxide, hydrogen gas, carbon dioxide, and water, at 100. Further, carbon efficiency may be improved according to a shift in the ratio of H₂ and CO (syngas) products by recycling the carbon dioxide side product, among other products, throughout the reaction processes; for example, carbon dioxide dry oxidative reforming of methane, at 102. Syngas formed among the reaction products may be recycled as fuel to drive methane pyrolysis, or as a hydrogen source for acetylene catalytic hydrogenation (at 104), or used in syngas conversions to other chemicals at 106. Consumption of the carbon dioxide side product in a methane dry oxidative reforming process may improve overall carbon efficiency of the process.

In some aspects, carbon dioxide, produced during the cracking processes, may be separated from the product stream and diverted to a separate reactor for conversion to syngas through methane oxidative dry reforming. After separation of carbon dioxide from the product stream, hydrogenation of acetylene to ethylene may provide the final products ethylene and syngas.

While it may be possible to re-direct generated carbon dioxide back to the pyrolysis (combustion/cracking) portion of the processes (to convert a portion of carbon dioxide to carbon monoxide and hydrogen gas), the ratio of hydrogen gas to carbon monoxide produced would be less than 2. Comparatively, subjecting the carbon dioxide to a separate dry oxidative reforming process may allow for production of a 2:1 hydrogen gas:carbon monoxide mixture from oxidative thermal conversion and a 1.5:1 ratio from the dry oxidative reforming process.

Moreover, where the carbon dioxide is consumed in a separate dry oxidative reforming process, there is no need for separation of carbon dioxide from carbon monoxide and hydrogen gas in the overall system product. That is, useful syngas and ethylene products need not be separated from a carbon dioxide side product in the system output. The disclosed methods herein separate the carbon dioxide product and utilize at least a portion of the carbon dioxide in a distinct process to shift the ratio of carbon monoxide and hydrogen gas in the overall system product.

### Oxidative Thermal Conversion

In various aspects, a hydrocarbon feed may be converted to acetylene and syngas via an oxidative thermal conversion, or pyrolysis, process to form at least acetylene, a carbon dioxide product and a first syngas product. Existing partial oxidation process may include a single step. For example, separately fed and preheated methane and oxygen feedstocks are mixed and combusted in a burner to provide the acetylene product. The acetylene product may be immediately cooled. Other processes however may include separate reaction zones, i.e., a zone dedicated to a first combustion of the hydrocarbon to supply the heat energy necessary to drive hydrocarbon pyrolysis in a second zone receiving a fresh hydrocarbon feed. Typically, the partial oxidation reactor system includes three major parts: a first portion (top) is a mixing zone with a special diffuser, the second portion (underneath) is a water-jacketed burner immediately followed by a reaction zone, and the third portion is a quenching zone using water or heavy oil as a coolant.

To produce acetylene from saturated hydrocarbons, energy must be supplied in large amounts at high temperatures. The conversion of methane to acetylene according to a thermal pyrolysis by combustion is a well-known process and has been presented in a number of patents as provided herein. Currently, there are two main variations for the processes. The first may involve combustion of a portion of a methane feed to generate heat sufficient according to an exothermic reaction (6) to convert a separate methane feed to acetylene (7).

2CH₄+_{3.5}O₂=CO₂+CO+4H₂O ΔH=−157 kcal/mol (6)

2CH₄=C₂H₂+3H₂ ΔH=45 kcal/mol (7)

The second variation may comprise oxidative pyrolysis of methane and selective oxidation reactions, each generating large amounts of heat reactions 8, 9, and 10.

2CH₄+_{1.5}O₂=C₂H₂+3H₂O ΔH=−41 kcal/mol (8)

CH₄+1.5O₂=CO+2H₂O ΔH=−103 kcal/mol (9)

CH₄+2O₂=CO₂+2H₂O ΔH=−174 kcal/mol (10)

Processing conditions may depend upon the net methane/oxygen (CH₄/O₂) ratio. The amount of oxygen feed delivered to the combustion process and present throughout the reaction system may dictate the reduction (reaction) or elimination of coke and soot formation. The O₂/CH₄ ratio may also affect the ratio of generated acetylene to synthesis gas, or syngas, comprising carbon monoxide (CO) and hydrogen gas (H₂). At a lower oxygen content for methane combustion, the combustion does not produce enough heat for cracking of methane to acetylene. With a higher oxygen content for methane combustion, a greater amount of methane is converted to carbon dioxide. A feed ratio (mole) for the oxidant and hydrocarbon feedstock can be from about to about 1.7 to about 1.8. For example, an oxygen to methane ratio may be fuel rich, that is, from about 1.7 to about 1.8.

As provided herein, a partial combustion of methane or a hydrocarbon feedstock to produce acetylene may be described as a single-stage burner process. The method may comprises two steps or stages which may occur almost simultaneously. As an example, in the combustion step, a portion of the methane or hydrocarbon feedstock may be burned with a quantity of an oxidant feedstream, such as oxygen, where the oxidant feedstream is insufficient for complete combustion. The oxygen feed may furnish heat at a temperature in the range of from about 1,200° C. to about 1,800° C. Because the combustion is incomplete, there remains a portion of the methane or the hydrocarbon feedstock which in turn may comprise the reaction component for the cracking reaction. Most of the remaining methane, or other hydrocarbon, may be cracked in a second step to acetylene by utilizing the heat energy available from the combustion step.

As a further example, the combustion of methane or a hydrocarbon feedstock to produce acetylene may proceed in a two-stage chamber or combustion reactor. In a first chamber, sufficient energy may be supplied to the chamber burner to combust a supply of hydrocarbon feedstock, or methane, and oxygen to produce high temperature gases. A separate feed of hydrocarbon, or methane, may be introduced to the first chamber and caused to flow to a second and burned with a quantity of oxygen.

The combustion of the methane or hydrocarbon feedstock may comprise a main reaction zone and a quenching zone. In the main reaction zone, the processes of combustion and cracking may occur. That is, the heated hydrocarbon feedstock, or methane, may be combusted providing sufficient energy to drive a subsequent cracking reaction and convert the methane to acetylene. As noted herein, the methane for cracking may be supplied by a separate hydrocarbon feed. In a further example, the methane for cracking may comprise a portion of the hydrocarbon feedstock that was not combusted. In the quenching zone, the acetylene product may be sufficiently cooled to prevent the decomposition of acetylene to its elemental carbon and hydrogen components.

The hydrocarbon feedstock may be the reaction component for the cracking and combustion operations described herein. In further examples, the hydrocarbon feedstock may comprise methane, heavy residue, natural case, natural gases, ethane, propane, naphtha, paraffins (e.g., alkanes (C1-C7) or saturated hydrocarbons, characterized by a general formula CₙH₂ₙ₊₂), unsaturated gases, or any combination thereof. In certain aspects, natural gas (e.g., having >85% methane) may be used as a hydrocarbon feedstock to produce ethylene. However, using natural gas in a thermal pyrolysis process may benefit from thermal exposure in a narrow temperature range to maximize yield of acetylene and ethylene. In some aspects, olefinic hydrocarbons such as ethane (ethylene), propene, butene, pentene, and/or hexene can be used, alone or in combination with other gases described.

### Separation

The hydrocarbon feed may undergo an oxidative thermal pyrolysis to form a product mixture comprising a gas stream of at least acetylene, carbon dioxide, hydrogen, water, and carbon monoxide. In various aspects, one or more separation processes may be employed to isolate gaseous components generated during the hydrocarbon combustion and cracking processes. The isolation of gaseous components of the product mixture may also be performed according to a number of gas separation techniques commonly practiced by those skilled in the art. For example, but not to be limiting, the isolation of unsaturated hydrocarbons may be achieved via a cold box separation, cryogenic processing, membrane separation, or pressure swing absorption.

In some aspects, carbon dioxide may be removed from the product mixture of gaseous components prior to separation of unsaturated hydrocarbons such as acetylene. The carbon dioxide product may be separated or diverted as a distinct process stream from the product mixture and converted to syngas via a methane dry oxidative reforming reaction according to the methods disclosed herein. As provided herein the separation of CO₂ from the combustion/cracking product mixture may be achieved by any known methods. In one example, adsorption processes, such as amine adsorption, may be used to separate carbon dioxide from the remaining combustion/cracking products. Amine adsorption may refer to the use of an amine sorbent to capture carbon dioxide gas.

Separation of unsaturated hydrocarbons, including acetylene, from the product mixture may also be performed according to a number of gas separation techniques commonly practiced by those skilled in the art. In one example, an isolated acetylene process stream may be obtained using an acetylene absorbing unit. In certain aspects of the present disclosure, the isolated acetylene may be dissolved in an appropriate solvent for hydrogenation to ethylene.

In further aspects, at least a portion of syngas can be separated from the product mixture to yield recovered synthesis gas, for example by cryogenic distillation. As might be appreciated by one of skill in the art, and with the help of this disclosure, the recovery of synthesis gas may be achieved as a simultaneous recovery of both H₂ and CO. At least a portion of the recovered syngas may be recycled back to process streams for hydrogenation of acetylene to ethylene according to the methods disclosed herein. Similarly, at least a portion of the recovered synthesis gas can be further converted to olefins (e.g., alkenes, characterized by a general formula CₙH₂ₙ). For example, the recovered synthesis gas can be converted to alkanes by a Fisher-Tropsch process, and the alkanes can be further converted by dehydrogenation into olefins.

### Dry Reforming Process

According to various aspects of the present disclosure, generated carbon dioxide may be separated from the combustion/cracking product mixture and converted to syngas via a methane dry oxidative reforming process. A mixture of methane (CH₄), oxygen (O₂) and carbon dioxide (CO₂) may be contacted with a suitable catalyst (i.e., a reforming catalyst) to form syngas.

Utilization of the generated carbon dioxide in a methane dry oxidative reforming process may improve carbon efficiency of the overall process of hydrocarbon conversion. In some examples, where the hydrocarbon feedstock comprises primarily methane, the pyrolysis product mixture may comprise between 15-30% carbon dioxide, on a water free basis. While it may be possible to divert carbon dioxide reactive products back to the pyrolysis portion to convert some carbon dioxide to carbon monoxide and hydrogen gas, the ratio of hydrogen gas to carbon monoxide produced would be less than 2. Separately subjecting the carbon dioxide to the dry reforming oxidative process may allow for production of a 2:1 hydrogen gas:carbon monoxide mixture from oxidative thermal conversion and a 1.5:1 ratio from the dry reforming process. Moreover, where the carbon dioxide is subjected to a separate dry reforming process, there is no need for separation of carbon dioxide from carbon monoxide and hydrogen gas in the product. The disclosed methods herein may divert carbon dioxide from the pyrolysis product mixture and subject the carbon dioxide to a separate process to shift the ratio of hydrogen gas and carbon monoxide in the overall product.

Carbon dioxide in the dry oxidative reforming of methane is a well-studied reaction that is of scientific and industrial importance. Compared to the endothermic reaction of methane dry reforming (11) to produce syngas, the process of methane dry oxidative reforming is exothermic and more energy efficient.

CH₄+CO₂=2CO+2H₂ ΔH=60 kcal/mol (11)

Dry oxidative reforming of methane may comprise the conversion of methane with carbon dioxide in an oxygen medium according to a combination of exothermic and endothermic processes. The overall reaction may be characterized as (12):

2CH₄+1/2O₂+CO₂=3CO+4H₂ (12)

In some aspects, the methane dry oxidative reforming process may comprise contacting a mixture of methane, oxygen, and carbon dioxide with a reforming catalyst. Here, endothermic dry reforming and exothermic methane oxidation can be performed in a single regime, which may provide an effective means to decrease the energy consumption during syngas synthesis. As an example, the H₂/CO ratio of the produced syngas composition may be approximately 1.4-1.8, which is highly advantageous for use of syngas in Fischer-Tropsch synthesis.

Oxidative dry reforming of the hydrocarbon feedstock comprising methane may be performed in the presence of a “reforming catalyst.” Useful reforming catalysts are also catalysts capable of converting carbon dioxide to syngas in the presence of oxygen. Reforming catalysts useful for oxidative dry reforming of a hydrocarbon feed include, but are not limited to, nickel/lanthanum(III)oxide (Ni/La₂O₃) catalyst; nickel/aluminum oxide (Ni/AI₂O₃) catalyst; and nickel/magnesium oxide-aluminum oxide (Ni/MgO-AI₂O₃) catalyst. The catalysts may be generated in situ. In a specific example, the reforming catalyst used in the process of the present disclosure is a Ni/La₂O₃ catalyst containing 5% nickel (Ni) on lanthanum(III) oxide (La₂O₃).

As an example, a process stream comprising CH₄, O₂ and CO₂ may be converted by catalytic dry reforming by contacting said feedstream with an Ni/La₂O₃ catalyst at a reaction temperature between about 650° C. to about 710° C. to produce a reformed gas that comprises at least CO and H₂ gases. The unconverted methane and carbon dioxide remaining in the gas phase may be less than about 5% by weight. The dry oxidative reforming process for the conversion of formed carbon dioxide may have a percent conversion of about 95%.

### Syngas

In various aspects, syngas may be formed among the products of the integrated reaction processes disclosed herein. As provided, a carbon dioxide process stream diverted from the pyrolysis product mixture may be converted to syngas via an integrated process of dry oxidative reforming. Syngas, or synthesis gas, may refer to gaseous mixture containing hydrogen (H₂) and carbon monoxide (CO), which may further contain other gas components like carbon dioxide (CO₂), water (H₂O), methane (CH₄), and nitrogen (N₂).

Syngas may be obtained through various chemical and thermochemical processes from almost any carbon source, such as oil, carbon, biomass, or biodegradable waste, but natural gas and low molecular weight hydrocarbons are the predominant starting materials. A conventional technology for producing syngas may comprise hydrocarbon steam reforming. Steam reforming involves the endothermic conversion of methane, or a hydrocarbon feedstock, and steam into hydrogen and carbon monoxide. Generally, steam reforming results in syngas gas having a H₂/CO molar ratio that is higher than the ratio needed for the synthesis of by-products, such as methanol or derivatives from the Fischer-Tropsch reaction. Industrially, the H₂/CO molar ratio is therefore typically adjusted.

In various aspects, syngas may be formed among the products of the integrated reaction processes disclosed herein. As provided herein, a carbon dioxide process stream diverted from the pyrolysis product mixture may be converted to syngas via an integrated process of dry oxidative reforming of methane. Syngas may also be formed in the cracking/combustion of methane provided herein.

Syngas generated among the products of the integrated processes herein may be recycled among the integrated processes. For example, at least a portion of the syngas generated from the cracking/combustion stage may be recycled as fuel back to the cracking/combustion stage. Where the syngas is recycled back to the cracking/combustion stage to generate heat energy, the amount of methane supplied to the combustion zone may be reduced. In this case, the ratio of oxygen to methane and syngas may be the same as when only methane was fed to combustion zone. In certain aspects, syngas is not recycled back to combustion zone. After use to facilitate the hydrogenation of acetylene, the syngas may be collected and used for additional products as well as for conversion to methanol. In a further example, a portion of the syngas may be used to facilitate hydrogenation of the generated acetylene. The effluent gas after hydrogenation of acetylene, generally contains more carbon monoxide and less hydrogen.

A process stream from which acetylene has been absorbed and within which syngas remains may be combined with a syngas process stream formed from the methane dry oxidative reforming reaction. Typically, syngas from the methane dry oxidative reforming reaction contains more hydrogen and less CO. The syngas maybe used to produce a wide range of additional products, such as higher alkanes and oxygenates by means of Fischer-Tropsch synthesis.

### Acetylene Hydrogenation

The integrated processes disclosed herein may include a selective hydrogenation of alkynes to alkenes. More specifically, the integrated processes may include a selective hydrogenation of acetylene to ethylene. In one aspect, the disclosed methods may comprise a liquid phase catalytic hydrogenation of acetylene.

While catalytic hydrogenation of acetylene to ethylene in gas phase is well-known, the gas-phase process is conventionally used to convert trace quantities, i.e., less than 2% of acetylene, produced during the production of ethylene by steam cracking of ethane or naphtha. Gas phase hydrogenation may not be appropriate in converting amounts of acetylene greater than 2% because of potential temperature run away scenarios. Hydrogenation is also known to occur in the liquid phase where fluids are easily conveyed or transported as liquids under reasonable temperature and pressure. In some aspects, a liquid phase hydrogenation of acetylene may be preferred. In a liquid phase acetylene catalytic hydrogenation, the catalyst may be substantially or completely wetted, thereby limiting access to the limiting reactant.

Useful processes for liquid phase selective catalytic hydrogenation of acetylene have been described. Alkynes, such as acetylene and/or acetylenic compounds, are absorbed from a gas or liquid stream using a non-hydrocarbon absorbent liquid. The absorbent liquid comprising the alkyne is contacted with one or more Group VIII catalyst mixtures to produce the alkene product, rather than an alkane.

In various aspects of the present disclosure, acetylene may be separated from a process stream of the pyrolysis product mixture which may comprise acetylene, CO, H₂, methane, and carbon dioxide. Acetylene may be absorbed from the pyrolysis product mixture by use of an appropriate solvent, such as a non-hydrocarbon absorbent liquid. Exemplary solvents may include, but are not limited to, n-methyl-2-pyrrolidone (NMP), tetrahydrofuran (THF), dimethylsulfoxide (DMSO), monomethylamine (MMA), and/or combinations thereof. In a specific example, the solvent is NMP. The solvent is typically capable of absorbing in the range of about between about 0.01 to 100 vol/vol acetylene and/or acetylenic compounds at standard conditions of temperature and pressure (STP) (i.e., a temperature of 273.15 kelvin (K) (0° C.) and an absolute pressure of 101.325 kiloPascals (kPa)). Any of the conventional techniques to accomplish the absorption that are known to those skilled in the art may be employed without departing from the scope of the disclosure.

The solvent including dissolved acetylene may be contacted with an appropriate hydrogenation catalyst. Exemplary hydrogenation catalysts include Group VIII or a mixture of Group VIII catalysts, which may be co-formulated with other metals such as those from Groups I through VII. The hydrogenation catalyst may preferably may be a supported catalyst comprising about 0.01% to 10% Group VIII metal or about 0.01% to 10% Group VIII metal and 0.01% to 10% Group I through Group VII metal. The catalyst may comprise Raney nickel, palladium on alumina, ruthenium on alumina, nickel arsenide on alumina, zinc oxide, zinc sulfide, and mixtures of one or more of the above, in addition to other catalysts as are known to those skilled in the art. The catalyst may also comprise palladium/gold on alumina (Al₂O₃) or the Lindlar catalyst (palladium on calcium carbonate and poisoned with lead or sulfur). In another aspect, the catalyst may preferably be palladium/gallium on alumina and/or palladium/indium on alumina and/or palladium/zinc on alumina. In a specific example, the hydrogenation catalyst comprises a 0.3% palladium-zinc aluminum oxide Pd—Zn/Al₂O₃ complex.

The solvent may be contacted with hydrogenation catalyst according to a number of processes known to one skilled in the art. For example, the solvent may be contacted with the hydrogenation catalyst(s) via slurry bubble column reactors, trickle bed reactors, three phase fluidized beds, fixed or moving bed reactors, riser reactors, fast-fluidized beds, or any other reaction system. Methods of contacting are appropriate so long as the reactant stream and catalyst are contacted under conditions suitable for hydrogenation and at a pressure and temperature sufficient to maintain the absorbent liquid in the liquid phase, with at least a portion of the acetylene contained in the absorbent being hydrogenated.

In certain aspects, a portion of hydrogen gas produced among the integrated processes described herein may be used in the catalytic hydrogenation of acetylene. Hydrogen may be furnished from among the integrated processes in a sufficient quantity to hydrogenate at least a portion of the absorbed acetylene. For example, hydrogen gas formed from the pyrolysis of methane may be used for downstream hydrogenation of acetylene to ethylene. In a further example, process streams comprising carbon monoxide CO and hydrogen gases (syngas) H₂ produced from the processes of methane pyrolysis and methane dry oxidative reforming may be combined and used to facilitate the catalytic hydrogenation disclosed.

Reactive products of the hydrogenation may include at least ethylene and a hydrogenation product, such as green oil. Green oil as used herein may refer to a mixture of high molecular weight oligomers of olefins formed in hydrogenation. In some examples, the hydrogenation product may be separated from the liquid phase and recycled back to the combustion/cracking stage of the integrated processes. Among the remaining reactive products, ethylene may be separated from syngas via conventional processes, such as distillation.

Absorption of acetylene from a process stream comprising the pyrolysis product mixture may be performed at a temperature of between about −17.78° C. and 204.4° C. (0° F. and 400° F.), and a pressure between about 1 pound per square inch absolute (psia) and 2000 psia. The size, capacity, and scope of any particular aspect of the disclosed process to be implemented may be determined following standard engineering practices well-known to those skilled in the art and following performance data presented herein.

### Systems

Various systems may make use of the integrated processes and methods described herein. A method of converting a hydrocarbon feedstock or feedstream may comprise causing combustion of a fuel (e.g., methane) to generate heat to drive cracking of a hydrocarbon feedstream to complex, unsaturated hydrocarbons. The method may also comprise separating a portion of products from the combustion and cracking of hydrocarbons for additional processing. The method may further comprise separating a reactive carbon dioxide product and subjecting the carbon dioxide to a dry oxidative reforming of methane process to form syngas. Acetylene may also be separated as a reactive product and catalytically hydrogenated in the liquid phase to provide ethylene. Remaining reactive products and components generated throughout the processes may be recycled to different stages throughout the processes to facilitate efficient conversion of the hydrocarbon feedstream.

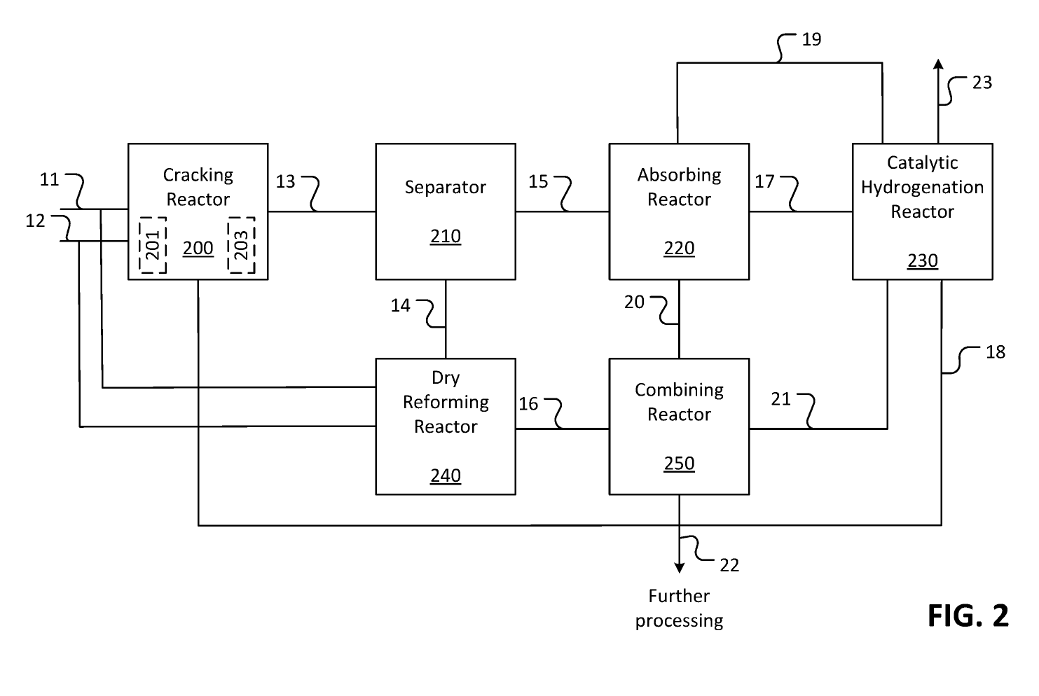

Referring now to FIG. 2, shown therein are certain processes for producing unsaturated C2 hydrocarbons in accordance with the present disclosure. In some aspects, impurities and contaminants may be first removed from an inlet feedstream such as hydrocarbon feedstream 11, which may primarily comprise methane. A portion of the hydrocarbon feedstream 11 may be conveyed to a cracking reactor 200 for pyrolysis with an oxidant feedstream 12. As is well known to those skilled in the art, cracking reactor 200 may comprise a single device or multiple devices. Each device may comprise one or more sections or zones. In the example shown in FIG. 2, the cracking reactor 200 may comprise a main reaction zone 201 and a quenching zone 203. Combustion and cracking of the hydrocarbon feedstream 11 may occur in the main reaction zone 201. The resultant combustion and cracking products may be cooled in the quenching zone 203 to provide a product mixture gas stream. The product mixture gas stream may be conveyed to a separator 210 schematically shown as output stream 13 of the cracking reactor 200.

As provided herein, the cracking reactor 200 may comprise one or more sections or zones dedicated to distinct processes. In one example, the cracking reactor 200 may comprise a main reaction zone 201 and a quenching zone 203. The main reaction zone 201 may comprise a combustion section or burner, which may be an in-line upstream burner, where the hydrocarbon feedstream 11 is burned, with the oxidant feedstream 12. The incoming hydrocarbon feedstream 11 may be pre-heated in pre-heaters (not shown) before it is heated to the preferred reaction temperature by direct heat exchange through combination with the hydrocarbon-combustion gas. The flame temperature of hydrocarbon feedstream 11 is preferably adequate to reach a desired reaction temperature preferably between 1200° C. to 2800° C., or from about 1800° C. to about 2000° C., with air or the oxidant (oxygen) or a combination of air and the oxidant. The addition of water or steam (not shown) to the main reaction zone 201 of the cracking reactor 200 may be used to lower and thereby control the combustion gas temperature.

In the cracking reactor 200, combustion of the hydrocarbon feedstream 11, in a burner may provide the energy sufficient for conversion of the hydrocarbon feedstream 11 to acetylene in the main reaction zone 201. The resultant combustion and conversion products may be cooled in the quenching zone 203 of the cracking reactor 200 to form the product mixture gas stream as outlet stream 13. The quenching may take place within about 1 to about 100 milliseconds (ms). The quenching zone 203 may achieve quenching of the combustion and conversion products by any of the methods known in the art including, without limitation, spraying a quench fluid such as steam, water, oil, or liquid product into a reactor quench chamber; conveying through or into water, natural gas feed, or liquid products; generating steam; or expanding in a kinetic energy quench, such as a Joule Thompson expander, choke nozzle, or turbo expander. Quenching may be accomplished in multiple steps using different means, fluids, or both. Accordingly, the quenching zone 203 may be incorporated within the cracking reactor 200, may comprise a separate vessel or device from the cracking reactor 200, or both.

The residence time of the combined combustion and cracking in the main reaction zone 201 of the cracking reactor 200 is sufficient to convert at least a portion of hydrocarbon feedstream 11 to at least acetylene, carbon monoxide, hydrogen gas methane and carbon dioxide compounds, and not so long as to allow significant further reactions to occur before quenching. In some examples, the residence time may be maintained under 100 ms or under 80 ms, to minimize coke formation. Residence times in excess of 0.1 ms or more than 0.5 ms are preferred to obtain sufficient conversion.

Adjustments may be made to the reaction temperature and pressure, and/or quenching after a desired residence time. In an example, the pressure of the hydrocarbon feedstream 11 may be maintained within the cracking reactor 200 between 1 bar and 20 bar (100 kiloPascals, kPa-2000 kPa) to achieve the product mixture as outlet stream 13.

The cracking reactor 100 may be configured to accommodate one or more feedstock streams. For example, the hydrocarbon feedstream 11 (FIG. 2) may include multiple hydrocarbon streams. The feedstock streams may include hydrocarbon combined with other gas components. Hydrocarbon feedstream 11 (FIG. 2), for example may include natural gas combined with other gas components including, but not limited to, hydrogen, carbon monoxide, carbon dioxide, and methane. In a further example, the cracking reactor 200 may have one or more oxidant feed streams 12 (FIG. 2), such as an oxygen stream and an oxygen-containing stream such as an air stream, which employ unequal oxidant concentrations for purposes of temperature or composition control.

In some aspects, insufficient oxidant for combustion may result in the formation of carbon monoxide. As an example, when insufficient oxygen via the oxidant feedstream 12 is introduced to the cracking reactor 200 to provide for complete combustion of either the hydrocarbon feedstream 11 intended as combustion gas or the combined stream of hydrocarbon feedstream 11 which serves as feed gas for cracking and a combustion gas, carbon monoxide may be formed. If formed, this carbon monoxide may be combined in whole or in part with the product mixture gases of outlet stream 13.

Also shown in FIG. 2, at least a portion of an outlet stream 13 from the cracking reactor 200 comprising at least acetylene, carbon monoxide, hydrogen, methane and carbon dioxide may be conveyed to a separator 210 for separation of the constituent gases. The separator 210 may comprise any appropriate gas separation method known to those with skill in the art. Carbon dioxide may be removed from process stream 13. In the separator 210, carbon dioxide may be separated from the process stream 13 by conventional means including, but not limited to, pressure swing absorption, membrane separation, cryogenic processing, and other gas separation techniques practiced by those skilled in the art.

At least a portion of outlet stream 14 of separator 210 comprising carbon dioxide may be conveyed to a dry reforming reactor 240. In the dry reforming reactor 240, carbon dioxide may be combined and combusted with at least a portion of a hydrocarbon feedstream 11 and at least a portion of the oxidant feed stream 12. Reactive products of the dry reforming reactor 240 may comprise at least carbon monoxide and hydrogen gas (syngas), shown as an outlet stream 16 of dry reforming reactor 240.

A separator 210 outlet stream 15 may comprise remaining gas components. In an example, the separator 210 outlet stream 15 may include at least acetylene, carbon monoxide, hydrogen gas, and unreacted hydrocarbon (methane). Outlet stream 15 may be directed to an absorbing reactor 220 for dissolution of acetylene into an appropriate solvent. At least a portion of acetylene may be absorbed by an appropriate non-hydrocarbon solvent thereby providing an acetylene process stream, outlet stream 17 from the absorbing reactor 220. The acetylene process stream 17 may be conveyed to a catalytic hydrogenation reactor 230 for a liquid phase catalytic hydrogenation of the acetylene gas to ethylene.

In a catalytic hydrogenation reactor, such as the catalytic hydrogenation reactor 230 at FIG. 2, the acetylene process stream 16 (in liquid phase) may be contacted with at least a hydrogenation catalyst to form ethylene gas. Reactive products of the catalytic hydrogenation reactor 230 may comprise at least a portion of a hydrogenation side product, or green oil as process outlet stream 18. Outlet stream 18 may be recycled to the cracking reactor 200 as fuel for the processes of combustion and cracking of hydrocarbon feedstream 11. The non-hydrocarbon solvent, such as NMP, may be recycled as outlet stream 19 from the catalytic hydrogenation reactor to the absorbing reactor to absorb acetylene gas.

In some aspects, syngas process streams may be combined. An outlet stream 16, from the dry reforming reactor 240 may comprise the reactive products of dry oxidative reforming of methane including, at least, carbon monoxide and hydrogen as syngas. A remaining components stream 20 may comprise at least carbon monoxide and hydrogen gas from which acetylene has been absorbed in the absorbing reactor 220. Outlet stream 16 and remaining components stream 20 may be combined, in for example, a combining reactor 250, to provide syngas process streams 21, 22. A portion of syngas may be recycled to the catalytic hydrogenation reactor so that at least a portion of hydrogen gas may be used in the hydrogenation of acetylene shown as process stream 21. In further examples, at least a portion of a syngas as process outlet stream 22 may be collected for further processing, such as syngas conversion reactions, including syngas to methanol or syngas to olefins. The unsaturated hydrocarbon product ethylene may be separated via distillation, for example, to provide outlet process stream 23.

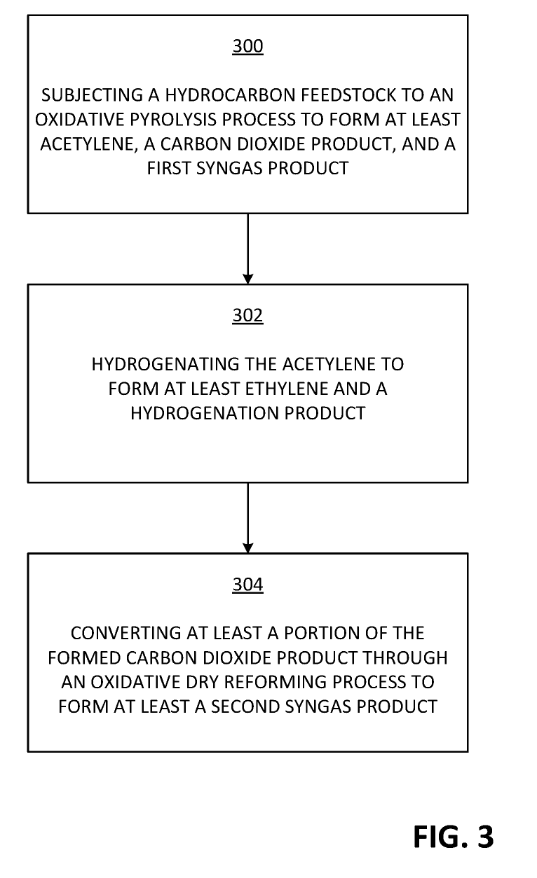

As provided in FIG. 3, in some aspects, a method of converting a hydrocarbon feedstock may comprise subjecting a hydrocarbon feedstock to an oxidative pyrolysis process. The hydrocarbon feedstock may be combusted with oxygen to form a product mixture comprising at least acetylene, a carbon dioxide product and a first syngas product at 300. The gaseous components of the product mixture may be separated or isolated for further processing to provide the unsaturated hydrocarbon product. Carbon dioxide may be diverted from the product mixture. The formed acetylene may be hydrogenated to form at least ethylene and a hydrogenation product, 302. At least a portion of the carbon dioxide product may be converted to at least a second syngas product through an oxidative dry reforming process at 304.

Other side products, including the hydrogenation product and the syngas products, originating from the hydrocarbon feedstock combustion and from the dry oxidative methane reforming reaction process, may be recycled throughout the methods disclosed herein. The syngas products and the hydrogenation product may be recycled as fuel to the process for the combustion of the hydrocarbon feedstock. In some examples, the syngas may be recycled to acetylene hydrogenation processes as a source of hydrogen. In further examples, the syngas products may be combined for processes of syngas conversions to useful chemicals including methanol.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Aspects

The disclosed systems and methods include at least the following aspects.

Aspect 1. A method of producing unsaturated C2 hydrocarbons comprising: subjecting a hydrocarbon feedstock to an oxidative pyrolysis process to form at least acetylene, a carbon dioxide product, and a first syngas product; hydrogenating the acetylene to form at least ethylene and a hydrogenation product; and converting at least a portion of the carbon dioxide product through an oxidative dry reforming process to form at least a second syngas product.

Aspect 2. A method of producing unsaturated C2 hydrocarbons consisting essentially of: subjecting a hydrocarbon feedstock to an oxidative pyrolysis process to form at least acetylene, a carbon dioxide product, and a first syngas product; hydrogenating the acetylene to form at least ethylene and a hydrogenation product; and converting at least a portion of the carbon dioxide product through an oxidative dry reforming process to form at least a second syngas product.

Aspect 3. A method of producing unsaturated C2 hydrocarbons consisting of: subjecting a hydrocarbon feedstock to an oxidative pyrolysis process to form at least acetylene, a carbon dioxide product, and a first syngas product; hydrogenating the acetylene to form at least ethylene and a hydrogenation product; and converting at least a portion of the carbon dioxide product through an oxidative dry reforming process to form at least a second syngas product.

Aspect 4. The method of any of aspects 1-3, wherein the first syngas product has a ratio of about 2:1 hydrogen gas to carbon monoxide and wherein the second syngas product approaches a ratio of 1.5 hydrogen gas to carbon monoxide.

Aspect 5. A method of producing unsaturated C2 hydrocarbons comprising: subjecting a hydrocarbon feedstock to an oxidative pyrolysis process to form at least acetylene, a carbon dioxide product, and a first syngas product; hydrogenating the acetylene to form at least ethylene and a hydrogenation byproduct; and converting at least a portion of the formed carbon dioxide byproduct through an oxidative dry reforming process to form at least a second syngas product, wherein the first syngas product approaches a ratio of 2:1 hydrogen gas to carbon monoxide and wherein the second syngas product approaches a ratio of 1.5 hydrogen gas to carbon monoxide.

Aspect 6. A method of producing unsaturated C2 hydrocarbons consisting essentially of: subjecting a hydrocarbon feedstock to an oxidative pyrolysis process to form at least acetylene, a carbon dioxide product, and a first syngas product; hydrogenating the acetylene to form at least ethylene and a hydrogenation byproduct; and converting at least a portion of the formed carbon dioxide byproduct through an oxidative dry reforming process to form at least a second syngas product, wherein the first syngas product approaches a ratio of 2:1 hydrogen gas to carbon monoxide and wherein the second syngas product approaches a ratio of 1.5 hydrogen gas to carbon monoxide.

Aspect 7. A method of producing unsaturated C2 hydrocarbons consisting of: subjecting a hydrocarbon feedstock to an oxidative pyrolysis process to form at least acetylene, a carbon dioxide product, and a first syngas product; hydrogenating the acetylene to form at least ethylene and a hydrogenation byproduct; and converting at least a portion of the formed carbon dioxide byproduct through an oxidative dry reforming process to form at least a second syngas product, wherein the first syngas product approaches a ratio of 2:1 hydrogen gas to carbon monoxide and wherein the second syngas product approaches a ratio of 1.5 hydrogen gas to carbon monoxide.

Aspect 8. The method of any of aspects 1-7, further comprising one or more separation processes to separate the carbon dioxide product from the acetylene and first syngas product, or to separate the acetylene from the first syngas product and the carbon dioxide product, or to separate the syngas from one or more of the carbon dioxide product and the acetylene.

Aspect 9. The method of aspect 8, wherein the one or more separation processes comprise an amine adsorption process.

Aspect 10. The method of aspect 8, wherein the one or more separation processes comprise pressure swing adsorption.

Aspect 11. The method of aspect 8, wherein the separation process comprises a cold box separation.

Aspect 12. The method of any of aspects 1-11, further comprising combining the first and the second syngas products and directing the first and the second syngas products to the hydrocarbon feedstock for the oxidative pyrolysis process.

Aspect 13. The method of any of aspects 1-7, further comprising combining the first and the second syngas products and directing the first and the second syngas products to a syngas conversion process.

Aspect 14. The method of any of aspects 1-11, further comprising combining the first and the second syngas products and directing the first and the second syngas products to promote hydrogenation of the acetylene.

Aspect 15. The method of any of aspects 1-14, wherein the hydrocarbon feedstock comprises saturated hydrocarbons.

Aspect 16. The method of any of aspects 1-14, wherein the hydrocarbon feedstock comprises methane, heavy residue, natural case, ethane, propane, naphtha, unsaturated gases, or a combination thereof.

Aspect 17. The method of any of aspects 1-16, wherein the oxidative pyrolysis process comprises a combustion of methane to generate heat and a conversion of a separate methane feed to form acetylene using the generated heat.

Aspect 18. The method of any of aspects 1-16, wherein the oxidative pyrolysis process comprises a combustion of methane in the presence of oxygen and a subsequent oxidation.

Aspect 19. The method of any of aspects 1-18, wherein the hydrogenating the acetylene comprises a liquid phase selective hydrogenation.

Aspect 20. The method of any of aspects 1-18, wherein the hydrogenating the acetylene comprises reacting the acetylene in a liquid phase in the presence of a catalyst.

Aspect 21. The method of aspect 20, wherein the catalyst is palladium based.

Aspect 22. The method of aspect 20, wherein the catalyst comprises a palladium-zinc aluminum oxide complex.

Aspect 23. The method of any of aspects 1-22, wherein the hydrogenation product comprises green oil.

Aspect 24. The method of any of aspects 1-23, further comprising directing the hydrogenation product to the hydrocarbon feedstock for oxidative pyrolysis.

Aspect 25. The method of any of aspects 1-24, wherein the oxidative dry reforming process comprises combusting the carbon dioxide product and methane in the presence of a reforming catalyst.

Aspect 26. The method of aspect 25, wherein the reforming catalyst facilitates the conversion of carbon dioxide product in the presence of oxygen.

Aspect 27. The method of any of aspects 25-26, wherein the reforming catalyst comprises a mixture of nickel oxide and lanthanum oxide, further comprising about 5% nickel on lanthanum oxide.

Aspect 28. The method of any of aspects 1-27, wherein the oxidative dry reforming process provides an amount of less than about 5% of remaining, unconverted methane and carbon dioxide.

Aspect 29. The method of any of aspects 1-28, wherein the oxidative dry reforming process for the conversion of formed carbon dioxide has a percent conversion of about 95%.

Aspect 30. A system for converting hydrocarbons to unsaturated C2-C3 hydrocarbons, the system comprising: a cracking reactor to effect at least a cracking or a combustion of a hydrocarbon feedstream to form a product mixture comprising at least acetylene, a carbon dioxide product, and a first syngas product; a separator to effect a separation among components of the product mixture; wherein acetylene is separated from the product mixture; carbon dioxide is separated from the product mixture, or a combination thereof; a hydrogenation reactor to effect at least a hydrogenation of the acetylene and a formation of a hydrogenation product; and a dry reforming reactor to effect at least conversion of carbon dioxide to a second syngas product.

Aspect 31. A system for converting hydrocarbons to unsaturated C2-C3 hydrocarbons, the system consisting essentially of: a cracking reactor to effect at least a cracking or a combustion of a hydrocarbon feedstream to form a product mixture comprising at least acetylene, a carbon dioxide product, and a first syngas product; a separator to effect a separation among components of the product mixture; wherein acetylene is separated from the product mixture; carbon dioxide is separated from the product mixture, or a combination thereof; a hydrogenation reactor to effect at least a hydrogenation of the acetylene and a formation of a hydrogenation product; and a dry reforming reactor to effect at least conversion of carbon dioxide to a second syngas product.

Aspect 32. A system for converting hydrocarbons to unsaturated C2-C3 hydrocarbons, the system consisting of: a cracking reactor to effect at least a cracking or a combustion of a hydrocarbon feedstream to form a product mixture comprising at least acetylene, a carbon dioxide product, and a first syngas product; a separator to effect a separation among components of the product mixture; wherein acetylene is separated from the product mixture; carbon dioxide is separated from the product mixture, or a combination thereof; a hydrogenation reactor to effect at least a hydrogenation of the acetylene and a formation of a hydrogenation product; and a dry reforming reactor to effect at least conversion of carbon dioxide to a second syngas product.

It is to be understood that the terminology used herein is for the purpose of describing particular aspects only and is not intended to be limiting. Although any methods and materials similar or equivalent to those described herein can be used in the practice or testing of the present disclosure, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior disclosure. Further, the dates of publication provided herein may be different from the actual publication dates, which can require independent confirmation.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, example methods and materials are now described.

### Definitions

It is to be understood that the terminology used herein is for the purpose of describing particular aspects only and is not intended to be limiting. As used in the specification and in the claims, the term “comprising” can include the embodiments “consisting of” and “consisting essentially of” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural equivalents unless the context clearly dictates otherwise. Thus, for example, reference to “a polycarbonate polymer” includes mixtures of two or more polycarbonate polymers.

As used herein, the term “combination” is inclusive of blends, mixtures, alloys, reaction products, and the like.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. “”“”It is generally understood, as used herein, that it is the nominal value indicated ±5% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

As used in the specification and the appended claims, the singular forms “a,” “an” and “the” include plural referents unless the context clearly dictates otherwise. Thus, for example, reference to “an injector” may include one or more injectors.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not. For example, the phrase “optionally substituted alkyl” means that the alkyl group can or cannot be substituted and that the description includes both substituted and un-substituted alkyl groups.

As used herein, “portion” may refer to a variable quantity ranging from none to all (i.e., 0% to 100%) with the specific quantity being dependent upon many internal factors, such as compositions, flows, operating parameters and the like as well as on factors external to the process such as desired products and by-products, or availability of electrical power, fuel, or utilities.

The term “hydrocarbon feedstock” or “hydrocarbon feedstream” as used herein refers to one or more feedstock or reaction streams that provide at least a portion of methane entering the cracking or combustion reactor as described herein or are produced from the reactor from the methane feed stream, regardless of whether further treatment or processing is conducted on such hydrocarbon feedstream. The “hydrocarbon feedstock” may include a methane feed stream, a reactor effluent stream, a desired product stream exiting a downstream hydrocarbon conversion process, or any intermediate or side product streams formed during the processes described herein. In certain systems, the hydrocarbon stream may be carried via a process stream line, which includes lines for carrying each of the portions of the process stream described above. The term “process stream” as used herein includes the “hydrocarbon stream” as described above, as well as it may include, alone or in combination, a carrier fluid stream, a fuel stream, an oxygen source stream, or any streams used in the systems and the processes described herein. The process stream may be carried via a process stream line, which includes lines for carrying each of the portions of the process stream described above.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

The following examples are provided to illustrate the compositions, processes, and properties of the present disclosure. The examples are merely illustrative and are not intended to limit the disclosure to the materials, conditions, or process parameters set forth therein.

### EXAMPLES

### Example 1

Example 1 describes combustion of methane and its pyrolysis at high temperature to acetylene and ethylene mixture as in FIG. 1 at 200. The processes were performed in a pilot scale reactor. The gas composition from the reactor is shown in Table 1:

TABLE 1
Processing conditions for combustion and cracking of methane

| Natural gas for | | | | | |
|---|---|---|---|---|---|
| cracking | Units | Run1 | Run2 | Run3 | Run4 |
| Natural Gas for | Ton/hour | 1.978E−2 | 1.978E−2 | 2.177E−2 | 2.208E−2 |
| Cracking | (lb/h) | (43.5) | (43.6) | (48.0) | (48.7) |
| Duty | kW | 3.265E5 | 3.265E5 | 3.261E5 | 3.247E5 |
| | (MBtu/h) | (1114) | (1114) | (1113) | (1108) |
| Natural Gas Fuel | Ton/hr | 2.521E−2 | 2.517E−2 | 2.527E−2 | 2.508E−2 |
| | (lb/h) | (55.6) | (55.5) | (55.7) | (55.3) |
| Oxygen | Ton/hr | 8.292E−2 | 8.264E−2 | 8.169E−2 | 8.060E−2 |
| | (lb/h) | (182.8) | (182.0) | (180.1) | (177.7) |
| O₂ | % | 88.4% | 88.0% | 87.2% | 86.5% |
| Stoichiometry | | | | | |

Cracked Gas composition on a water free basis

| | | | | | |
|---|---|---|---|---|---|
| H₂ | mol % | 35.30 | 30.90 | 33.59 | 33.44 |
| CO₂ | mol % | 19.64 | 20.39 | 19.67 | 19.19 |
| CH₄ | mol % | 14.46 | 13.27 | 17.23 | 18.88 |
| C₂H₄ | mol % | 0.48 | 1.58 | 0.55 | 0.63 |
| C₂H₂ | mol % | 4.58 | 5.87 | 4.36 | 4.09 |
| CO | mol % | 24.09 | 25.76 | 22.85 | 21.90 |

Performance metrics of the cracker

| | | | | | |
|---|---|---|---|---|---|
| Conversion of | % C | 51.3% | 59.1% | 45.8% | 41.5% |
| cracking reactor | | | | | |
| feed (excludes fuel) | | | | | |
| Cracking (C2 (as | % C | 34.0% | 45.6% | 30.7% | 29.2% |
| C₂H₄ + C₂H₂) Yield) | | | | | |
| Acetylene Yield | % C | 30.7% | 35.9% | 27.3% | 25.3% |

Units are defined as follows: lb/hr are pounds per hour; ton/hr are tons per hour; MBtu/h are one thousand-British thermal units per hour; and kW are kiloWatts.

### Example 2

Example 2 comprises oxidative cracking of C2-C3 hydrocarbons where naphtha (hydrocarbon stream from crude oil) replaces natural gas (primarily methane) at the same reactor and process conditions. The only difference of the process in case of natural gas and other hydrocarbons is in product distribution where acetylene:ethyelene (C₂H₂:C₂H₄) ratio changes.

TABLE 2
Processing conditions for combustion and cracking of naphtha

| CRACKING | Units | ETHANE | PROPANE | NAPHTHA |
|---|---|---|---|---|
| Cracking feed | Ton/hour | 3.202E−2 | 3.180E−2 | 3.171E−2 |
| | (lb/h) | (70.6) | (70.1) | (69.9) |
| Duty | kW | 2.696E5 | 2.693E5 | 2.731E5 |
| | (MBtu/h) | (920) | (919) | (932) |
| Natural Gas | Ton/hr | 2.059E−2 | 2.059E−2 | 2.091E−2 |
| Fuel | (lb/h) | (45.4) | (45.4) | (46.1) |
| Oxygen | Ton/hr | 6.754E−2 | 6.740E−2 | 6.736E−2 |
| | (lb/h) | (148.9) | (148.6) | (148.5) |
| O2 | % | 87.2% | 87.1% | 85.9 |
| Stoichiometry | | | | |

Cracked Gas composition on a water free basis

| | | | | |
|---|---|---|---|---|
| H₂ | mol % | 36.61 | 30.66 | 26.94 |
| CO₂ | mol % | 18.98 | 20.36 | 24.39 |
| CH₄ | mol % | 5.62 | 11.15 | 8.06 |
| C₂H₄ | mol % | 12.19 | 10.75 | 12.95 |
| C₂H₂ | mol % | 6.86 | 7.65 | 7.1 |
| CO | mol % | 15.68 | 16.82 | 16.36 |

Performance metrics of the cracker

| | | | | |
|---|---|---|---|---|
| Conversion of | % C | 78.0% | 77.4% | 84.9% |
| cracking | | | | |
| reactor feed | | | | |
| Cracking (C2 | % C | 69.9% | 63.2% | 62.5% |
| Yield) | | | | |
| Acetylene Yield | % C | 25.2% | 26.3% | 22.1% |
| Total yield | | 44.2% | 40.2% | 39.8% |

### Example 3

Example 3 provides experimental conditions for the dry oxidative reforming of methane CH₄+CO₂+O₂ as illustrated in FIG. 2 at the dry reforming reactor 240. Methane and oxygen are combined and combusted with carbon dioxide separated from the combustion and cracking products of the cracking reactor. The dry oxidative reforming reaction was performed at a temperature of 670° C. and space velocity (volumetric flow rate of feed divided by the volume of the catalyst) 2000 per hour (h⁻¹); at a temperature of 700° C. and space velocity 1600 h−1; at a temperature of 730° C. and a space velocity of 1800 h⁻¹. The catalyst is 5% Ni on Al₂O₃ and catalyst loading 5 ml with the methane to carbon dioxide and methane to oxygen ratios at: CH₄/CO₂=2, CH₄/O₂=2.5. The results of the reforming processes are presented in Table 3.

TABLE 3
Processing conditions and output of methane dry oxidative reforming

| | CH₄ Conversion | CO₂ conversion | O₂ conversion | H₂/CO |
|---|---|---|---|---|
| Conditions | (% mol) | (% mol) | (% mol) | ratio |
| 670° C./2000 h⁻¹ | 70 | 68.2 | 93 | 1.5 |
| 700° C./1600 h⁻¹ | 74.0 | 85.1 | 96.2 | 1.4 |
| 730° C./1800 h⁻¹ | 76.5 | 87.6 | 98.2 | 1.2 |
| 750° C./2400 h⁻¹ | 82 | 91 | 99.5 | 1.0 |

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All cited patents, patent applications, and other references are incorporated herein by reference in their entirety. However, if a term in the present application contradicts or conflicts with a term in the incorporated reference, the term from the present application takes precedence over the conflicting term from the incorporated reference. While typical aspects have been set forth for the purpose of illustration, the foregoing descriptions should not be deemed to be a limitation on the scope herein. Accordingly, various modifications, adaptations, and alternatives can occur to one skilled in the art without departing from the spirit and scope herein.

1. A method of producing unsaturated C2 hydrocarbons comprising:
subjecting a hydrocarbon feedstock to an oxidative pyrolysis process to form at least acetylene, a carbon dioxide product, and a first syngas product;
hydrogenating the acetylene to form at least ethylene and a hydrogenation product; and
converting at least a portion of the carbon dioxide product through an oxidative dry reforming process to form at least a second syngas product.

2. The method of claim 1, wherein the first syngas product approaches a ratio of about 2:1 hydrogen gas to carbon monoxide and wherein the second syngas product has a ratio of about 1.5 hydrogen gas to carbon monoxide.

3. The method of any of claim 1, further comprising one or more separation processes to separate the carbon dioxide product from the acetylene and first syngas product, or to separate the acetylene from the first syngas product and the carbon dioxide product, or to separate the syngas from one or more of the carbon dioxide product and the acetylene.

4. The method of claim 3, wherein the one or more separation processes comprise an amine adsorption process.

5. The method of claim 3, wherein the one or more separation processes comprise pressure swing adsorption.

6. The method of claim 1, further comprising combining the first and the second syngas products and directing the first and the second syngas products to the hydrocarbon feedstock for the oxidative pyrolysis process.

7. The method of claim 1, further comprising combining the first and the second syngas products and directing the first and the second syngas products to promote hydrogenation of the acetylene.

8. The method of claim 1, wherein the hydrocarbon feedstock comprises saturated hydrocarbons.

9. The method of claim 1, wherein the hydrocarbon feedstock comprises methane, heavy residue, natural case, ethane, propane, naphtha, unsaturated gases, or a combination thereof.

10. The method of claim 1, wherein the oxidative pyrolysis process comprises combustion of methane to generate heat and a conversion of a separate methane feed to form acetylene using the generated heat.

11. The method of claim 1, wherein the oxidative pyrolysis process comprises a combustion of methane in the presence of oxygen and a subsequent oxidation.

12. The method of claim 1, wherein the hydrogenating the acetylene comprises a liquid phase selective hydrogenation.

13. The method of claim 1, wherein the hydrogenating the acetylene comprises reacting the acetylene in a liquid phase in the presence of a catalyst.

14. The method of claim 13, wherein the catalyst comprises a palladium-zinc aluminum oxide complex.

15. The method of claim 1, further comprising directing the hydrogenation product to the hydrocarbon feedstock for oxidative pyrolysis.

16. The method of claim 1, wherein the oxidative dry reforming process comprises combusting the carbon dioxide product and methane in the presence of a reforming catalyst.

17. The method of claim 16, wherein the reforming catalyst facilitates conversion of carbon dioxide product in the presence of oxygen.

18. The method of claim 1, wherein the first and the second syngas product may be combined to form a feedstock of a syngas conversion process.

19. The method of claim 1, wherein the oxidative dry reforming process for the conversion of carbon dioxide product may have a percent conversion of about 95%.

20. A system for converting hydrocarbons to unsaturated C2-C3 hydrocarbons, the system comprising:
a cracking reactor to effect at least a cracking or a combustion of a hydrocarbon feedstream to form a product mixture comprising at least acetylene, a carbon dioxide product, and a first syngas product;
a separator configured downstream from the cracking reactor to effect a separation among components of the product mixture; wherein acetylene is separated from the product mixture; carbon dioxide is separated from the product mixture, or a combination thereof;
a hydrogenation reactor configured downstream from the separator to effect at least a hydrogenation of the acetylene and a formation of a hydrogenation product; and
a dry reforming reactor configured subsequent to the separator to effect at least conversion of carbon dioxide to a second syngas product.
